(19) United States
(12) Patent Application Publication
(10) Pub. No.: US 2026/0325730 A1
(43) Pub. Date: Oct. 22, 2026

(54) HEMODIALYSIS TREATMENT SYSTEM WITH VASCULAR ACCESS DEVICE MONITORING

(71) Applicant: Fresenius Medical Care Deutschland GmbH, Bad Homburg (DE)

(72) Inventors: Luca Neri, Bad Homburg (DE); Alessandro Bregoli, Bad Homburg (DE); Andreas Maierhofer, Bad Homburg (DE); Pascal Kopperschmidt, Bad Homburg (DE); Francesco Bellocchio, Bad Homburg (DE)

(73) Assignee: Fresenius Medical Care Deutschland GmbH, Bad Homburg (DE)

(21) Appl. No.: 19/634,969

(22) Filed: Mar. 31, 2026

(30) Foreign Application Priority Data
Apr. 17, 2025 (EP) 25171409.3

(51) Int. Cl. A61M 1/36; A61M 1/14; G16H 20/40

(52) U.S. Cl. CPC A61M 1/3656; A61M 1/15632; G16H 20/40; A61M 2205/502; A61M 2230/30

(57) ABSTRACT

A hemodialysis system includes: a hemodialysis treatment machine configured to provide a hemodialysis treatment for a patient, wherein the hemodialysis treatment includes using a dialyzer to remove urea from extracorporeal blood of the patient, and wherein the extracorporeal blood is obtained from the patient via a vascular access device of the patient; one or more measuring devices configured to measure one or more measured values corresponding to the hemodialysis treatment; and a computer system configured to: obtain a plurality of parameter values, wherein the plurality of parameter values include the one or more measured values and/or one or more calculated values calculated from the one or more measured values; predict a probability of failure of the vascular access device of the patient with a predetermined time period or within a predetermined number of treatment sessions; and output the predicted probability.

### CROSS-REFERENCE TO RELATED APPLICATION

Priority is claimed to European Patent Application No. EP 25171409.3, filed on Apr. 17, 2025, the entire disclosure of which is hereby incorporated by reference herein.

### TECHNICAL FIELD

The present invention relates to a computer-implemented method for predicting a patient's fistula or vascular access failure, in particular venous access failure, a computer system, and a hemodialysis treatment apparatus. Furthermore, it relates to a digital storage medium, a computer program product, and a computer program.

### BACKGROUND

For patients with chronic kidney disease (CKD), renal replacement therapy (RRT) is an essential treatment. Hemodialysis is one of the most commonly used forms of RRT and requires well-functioning vascular access for administration. An arteriovenous fistula (AVF) is one of the most commonly used types of vascular accesses, especially in Europe. As a result, significant efforts have been made to ensure the survival and patency of AVFs. Medical literature suggests that physical examination is an effective and inexpensive method for monitoring the AVFs. This is, however, time-consuming. The same applies for so-called arteriovenous (AV) grafts (AV-grafts), which resemble arteriovenous fistulas in many ways but encompass graft material (made from polytetrafluoroethylene (PTFE) or the like). Since such grafts may encounter identical or similar problems as arteriovenous fistulas, such grafts are considered hereinafter as well. Also, some patients are treated-mostly in acute care wards-using a central venous catheter or line. Those accesses to the patient's venous system are also considered hereinafter.

### SUMMARY

In an exemplary embodiment, the present application provides a hemodialysis system. The hemodialysis system includes: a hemodialysis treatment machine configured to provide a hemodialysis treatment for a patient, wherein the hemodialysis treatment includes using a dialyzer to remove urea from extracorporeal blood of the patient, and wherein the extracorporeal blood is obtained from the patient via a vascular access device of the patient; one or more measuring devices configured to measure one or more measured values corresponding to the hemodialysis treatment; and a computer system configured to: obtain a plurality of parameter values, wherein the plurality of parameter values include the one or more measured values and/or one or more calculated values calculated from the one or more measured values; predict a probability of failure of the vascular access device of the patient with a predetermined time period or within a predetermined number of treatment sessions; and output the predicted probability.

In another exemplary embodiment, the present application provides a vascular access monitoring method for a hemodialysis system. The method includes providing, by a hemodialysis treatment machine, a hemodialysis treatment for a patient, wherein the hemodialysis treatment includes using a dialyzer to remove urea from extracorporeal blood of the patient, and wherein the extracorporeal blood is obtained from the patient via a vascular access device of the patient; measuring, by one or more measuring devices, one or more measured values corresponding to the hemodialysis treatment; obtaining, by a computer system, a plurality of parameter values, wherein the plurality of parameter values include the one or more measured values and/or one or more calculated values calculated from the one or more measured values; predicting, by the computer system, a probability of failure of the vascular access device of the patient with a predetermined time period or within a predetermined number of treatment sessions; and outputting, by the computer system, the predicted probability.

In yet another exemplary embodiment, the present application provides one or more non-transitory computer-readable mediums having processor-executable instructions stored thereon for vascular access monitoring for a hemodialysis system. The processor-executable instructions when executed, facilitate performance of the following: providing, by a hemodialysis treatment machine, a hemodialysis treatment for a patient, wherein the hemodialysis treatment includes using a dialyzer to remove urea from extracorporeal blood of the patient, and wherein the extracorporeal blood is obtained from the patient via a vascular access device of the patient; measuring, by one or more measuring devices, one or more measured values corresponding to the hemodialysis treatment; obtaining, by a computer system, a plurality of parameter values, wherein the plurality of parameter values include the one or more measured values and/or one or more calculated values calculated from the one or more measured values; predicting, by the computer system, a probability of failure of the vascular access device of the patient with a predetermined time period or within a predetermined number of treatment sessions; and outputting, by the computer system, the predicted probability.

### BRIEF DESCRIPTION OF THE DRAWINGS

Exemplary embodiments of the present application will be described in even greater detail below based on the exemplary figures. The application is not limited to the exemplary embodiments. All features described and/or illustrated herein can be used alone or combined in different combinations in embodiments of the application. Features and advantages of various embodiments of the present application will become apparent by reading the following detailed description with reference to the attached drawings which illustrate the following:

FIG. 1 shows a schematically simplified fluid line diagram of a hemodialysis treatment apparatus according to the present invention in an exemplary embodiment; and

FIG. 2 shows a schematically simplified flowchart of a computer-implemented method according to the present invention in an exemplary embodiment.

### DETAILED DESCRIPTION

The present application includes example embodiments pertaining to arteriovenous fistulas (AVFs), but it is not limited thereto. The present application applies to vascular access devices generally, including AVFs, arteriovenous (AV) grafts, central venous catheters and/or any other type of vascular access or catheter.

Exemplary embodiments of the present application provide a computer-implemented method for predicting a patient's fistula failure, a computer system, a hemodialysis treatment apparatus, a digital storage medium, a computer program product, and a computer program.

According to the present invention, a computer-implemented method for predicting a patient's fistula failure or for assessing the probability of the fistula to fail is provided. Thereby, the probability to fail may be considered, e.g., with regard to a preset time period or to the course of pre-determined number of treatment sessions, or the like.

The computer-implemented method comprises an inputting of a plurality of parameter values into a computer system or a component thereof. Some of these values may have been measured during one or more dialysis treatment sessions while treating the patient using a hemodialysis treatment apparatus or in connection with such dialysis treatment sessions, respectively. Additionally or alternatively, some of these values may be or may have been calculated based on or derived from values having been measured during one or more dialysis treatment sessions while treating the patient using the hemodialysis treatment apparatus.

The computer-implemented method further comprises predicting or assessing the probability or estimated probability of an event predefined as a fistula failure within a predetermined time period. This time period may start as of the time of the prediction or as of the time of executing/carrying out, in particular starting, of this method. This time period may cover a predetermined number of, preferably subsequent, treatment sessions.

The computer-implemented method further comprises the predicted probability or the result of the assessment being output.

The predicted probability may in some embodiments be considered the target variable or the dependent variable in statistics. A target variable is the outcome one aims to predict or explain using, e.g., a machine learning model. A target variable is the variable that is estimated or classified based on the input values. The result of the assessment may be a simple “true” or “false”, e.g., with respect to a probability hypothesis.

In some embodiments, outputting the predicted probability or the result of the assessment may include information leaving the computer system on which a method according to the invention is carried out. It may, for example be displayed, sent, printed, or the like such that a person or an apparatus external to the computer system is provided with the predicted probability or the result. In some embodiments, outputting the predicted probability or the result of the assessment may include effecting changes in the control of an hemodialysis treatment apparatus, e.g. via a component for treating the patient.

According to the present invention, a computer system is provided that is programmed for carrying out/executing the computer-implemented method according to the present invention.

Furthermore, according to the present invention, a hemodialysis treatment apparatus is provided. The hemodialysis treatment apparatus comprises components for carrying out a hemodialysis treatment and a control unit for controlling the components.

The apparatus further comprises the computer system according to the present invention or is in signal communication with it. Alternatively, it is prepared for being in signal communication with it.

A digital, particularly non-volatile storage medium, according to the present invention, e.g. in the form of a machine readable carrier, e.g. in the form of a diskette, floppy disk, memory card, CD, DVD EPROM, FRAM (Ferroelectric RAM) or SSD (Solid-State-Drive), e.g. with electronically or optically readable control signals may interact with a computer system and/or interact with the controller of a hemodialysis treatment apparatus.

A computer program product according to the present invention comprises a volatile or transient program code or one stored on a machine-readable carrier or a signal wave, with which a computer system is programmed and/or the controller of a hemodialysis treatment apparatus is programmed when the computer program product runs on a computer.

A computer program product can be, for example, understood according to the present invention as a computer program stored on a carrier, an embedded system being a comprehensive system with a computer program (e.g., an electronic device with a computer program), a network of computer implemented computer programs (e.g. client/server-system, a cloud computing system etc.), or a computer on which a computer program is loaded, runs, is stored, is being executed or developed.

The term “machine readable carrier” as used herein, refers in certain embodiments of the present invention to a carrier, which contains data or information interpretable by software and/or hardware. The carrier may be a data carrier, such as a diskette, a CD, DVD, a USB stick, a flashcard, an SD card or the like, as well as any other storage referred to herein or any other storage medium referred to herein.

A computer program according to the present invention comprises a program code with which, when executed on a computer, a computer system is programmed and/or with which the controller of a hemodialysis treatment apparatus is programmed.

Embodiments according to the present invention may comprise and/or consist of one or several of the features mentioned supra and/or in the following. Thereby, these features may be combined unless a particular combination would be understood to be impossible from context or based on the understanding of a person of ordinary skill in the art.

In all of the aforementioned and following statements, the use of the expression “may be” or “may have” and so on, is to be understood synonymously with “preferably is” or “preferably has”, and so on respectively, and is intended to illustrate an embodiment according to the present invention.

Whenever an embodiment is mentioned herein, it represents an exemplary embodiment according to the present invention and is not to be understood as limiting.

When method steps are mentioned herein, then the devices according to the present invention are in several embodiments configured in order to execute, in any combination, one or several of these method steps, in particular when these are automatically executable steps, or in order to accordingly control corresponding apparatuses which preferably correspond with their names to the designation of the respective method step (for example, “determining” as a method step and “apparatus for determining” for the apparatus) and which may likewise be part of the apparatus(es) according to the present invention or connected thereto in signal communication.

When programmed or configured is mentioned herein, then these terms may in some embodiments be interchangeable.

When a signal communication or communication connection between two components is mentioned herein, this may be understood to mean a connection that exists during use. It may also be understood that a preparation for such a (wired, wireless or otherwise implemented) signal communication exists, for example by coupling both components, for example by pairing, etc.

Pairing is a process that takes place in connection with computer networks in order to establish an initial link between computer units for the purpose of communication. The best-known example of this is the establishing of a Bluetooth connection, by which various devices (e.g. smartphone, headphones) are connected to each other. Pairing is sometimes also referred to as bonding.

When method steps are mentioned herein, then it may be provided in several embodiments according to the system described herein that these steps are carried out prior to or after a treatment of the patient, for instance during the time when the patient is not connected, e.g. via the extracorporeal blood circuit or the like, to the apparatus or that a treatment has not begun yet or is already completed.

In some embodiments, the treatment of the patient may be completed or may not have started yet, when, e.g., the patient is not connected to the hemodialysis treatment apparatus, e.g., because their vascular system is not connected to an extracorporeal blood circuit in fluid communication, or when no blood or no liquid is returned into their vascular system, in particular not at the relevant point in time and/or not anymore in the further course of the relevant treatment session.

The computer system may be, or comprise, a control device or closed-loop control device that may prompt the execution of all or substantially all of the method steps. The method according to the present invention may substantially or completely be executed by the control device or closed-loop control device. It may be partly executed by the control device or closed-loop control device. Such method steps may include machine-induced or automatic method steps.

In some embodiments the verbs “turn”, “convert”, “change”, “transform” or “reprogram” may be used interchangeably.

In several embodiments of the computer-implemented method according to the present invention, the values of the parameters which are input comprise or consist of, at least two or more of, the following ones:
clearance;
sum pressure;
clearance/blood flow-ratio; or
clearance/reference clearance-ratio.

The clearance is the capacity of the dialyzer to clean the blood of urea, or is the urea removal rate, and can be calculated based on urea measurements, preferably in as a in-vivo clearance measurement. For example, the Online Clearance Monitoring (OCM®) system by Fresenius Medical Care, Germany, integrated, e.g., into their therapy system 5008, intra-dialytically measures the effective in-vivo urea clearance achieved during the treatment session.

To determine the clearance, a control and evaluation unit may influence the dialysate composition to induce a change of the conductivity at the dialyzer inlet, evaluate the “response” at the dialyzer outlet, and gather a value for the electrolyte clearance, which is representative of the urea clearance. There are other methods for measuring clearance, such as spectroscopy, which do not require inducing changes to the dialysate properties or composition.

Generally, for determining the clearance, a property of the used dialysate fluid is preferably measured. However, the property of interest does not necessarily have to be urea. Rather, the findings related the clearance of the property or urea may be used for determining the clearance in general or of interest.

EP 0845273 A1 discloses a method for determining the clearance (also referred to as dialysance D therein) for a mathematical determination of the clearance of the method or the system, in particular the hemofilter, used when treating the patient via dialysis. The same holds true for WO 2023/062047A1. The respective disclosures of EP 0845273A1 and WO 2023/062047A1 are incorporated herein by way of reference, both regarding the methods and the devices disclosed there.

In some embodiments, the reference clearance is an expected clearance based on treatment parameters like inter alia mass transfer-area coefficient (k0A) of the used dialyzer, blood flow rate of the extracorporeal blood circuit, dialysis fluid flow rate and treatment mode (hemodialysis, hemodiafiltration with post or pre dilution etc.), as disclosed in WO 2023/062047A1, while the clearance is the measured clearance of the actual treatment as determined e.g. as described in EP 0845273A1.

In certain embodiments, the following formula, found and explained in more detail in WO 2023/062047A1, is used for calculating the reference clearance Kref.

$${K}_{ref} = \frac{{Q}_{b}}{{Q}_{b} + κ {Q}_{s}} ( {D}_{diff} \frac{{Q}_{b} - {Q}_{f} - ( 1 - κ ) {Q}_{s}}{{Q}_{b} + κ {Q}_{s}} + {Q}_{f} + {Q}_{s} )$$

$$κ = \{ \begin{matrix}0 & HD , HDF - post \\ 1 & HDF - pre\end{matrix}$$

with
Qb being the blood flow rate
Qs being the substitute fluid flow rate
Qf being the ultrafiltration rate
Ddiff being the diffusive part of Clearance K
HD referring to hemodialysis
HDF-post referring to hemodiafiltration with post-dilution
HDF-pre referring to hemodiafiltration with pre-dilution.

The concept of measuring the clearance is described in DRUKKER, PARSONS and MAHER, “Replacement of Renal Function by Dialysis”, 5th edition, Kluwer Academic Publishers, Dordrecht/Boston/London 2004, ISBN 1-4020-0083-9, see in particular page 280, 414f and chapter 22. The respective disclosure thereof is incorporated herein by way of reference.

The sum pressure is the sum of the, e.g. simultaneously, measured arterial pressure and venous pressure, e.g. the pressure measured by sensors already present within the hemodialysis treatment apparatus.

The clearance/blood flow-ratio is the clearance divided by the blood flow or vice versa, and the clearance/reference clearance-ratio is the clearance divided by a reference clearance, or vice versa. The reference clearance may be pre-set or stored or looked-up from, e.g. a table.

There are several methods for determining the reference clearance: based on a model of the dialysis system (including, e.g., the dialyzer and the patient with recirculation), based on previous measurements, estimates from comparable cohorts, and others.

In general, the reference clearance, as referred to herein, may be derived at using the same methods or devices as used for deriving at the clearance as referred to herein.

Patent application WO 2023/062047 A1 discloses a number of methods for determining or estimating a clearance or a reference clearance, and further refers to additional methods not or not fully described in said patent application, all of which can be used for determining or estimating a clearance and/or a reference clearance for the purpose of this invention. WO 2023/062047 A1 is incorporated herein by way of reference.

In particular, reference values can be patient-specific, derived from current or past treatments. Alternatively, global, center-specific, or organization-specific values can also be used. These values may be available locally or distributed (e.g., in the cloud) and can be made available for analysis before or during treatment.

In some embodiments, the dynamic of the parameters during a treatment session of interest is not taken into account, but, for example, the median value of said parameter values measured during or calculated with respect to the treatment session.

In several embodiments, the predicted probability is output to a printer, a display, a handheld device of any kind, e.g. a smartphone, a tablet etc., and/or to the hemodialysis treatment apparatus.

In some embodiments, an artificial intelligence model, e.g., a machine learning model, is used for predicting the probability.

In several embodiments, a logistic regression model is used. This model estimates the probability of failure based on a linear combination of the input variables. Alternatively, a gradient boosting framework based on decision tree classifiers is used for predicting the probability, e.g. the LightGBM algorithm as shown in Guolin Ke et al. “LightGBM: A Highly Efficient Gradient Boosting Decision Tree” in: Advances in Neural Information Processing Systems, Ed. by I. Guyon et al, Vol. 30, Curran Associates, Inc., 2017. This is a more sophisticated model, if compared with the logistic regression, capable to deal with non-linear relation between the input variables and the target.

Experiments by the inventors have shown that the two above-mentioned approaches identify risk class with similar risk of failure. The main difference is observed in the high risk class (the 0.1% hemodialysis treatment sessions with the highest risk of failure), where the logistic regression is more effective in identify fistulas that will fail within a month.

In several embodiments, the computer-implemented method encompasses outputting suggestions regarding medical or surgical treatment based on the predicted probability or the result.

In some embodiments of the hemodialysis treatment apparatus, the control unit comprises or consists of the computer system, or vice versa.

In several embodiments, the hemodialysis treatment apparatus further comprises measuring devices for measuring the parameters the values of which are input. Additionally or alternatively, the hemodialysis treatment apparatus comprises a calculating device for calculating a value of at least one of the parameters.

In some embodiments, the hemodialysis treatment apparatus is a self-contained system. It may carry out the computer-implemented method according to the invention. In particular, it may measure the parameter values at issue or the values the parameter of interest are based on.

In some embodiments, the control unit is programmed to control the hemodialysis treatment apparatus during treatment based on the predicted probability output.

For example, in some embodiments, the control unit is programmed to limit the allowable blood flow rate that a doctor may set for treatment, both during an ongoing treatment session and for a treatment session yet to be started.

Also, in certain embodiments, the control unit is programmed to adjust automatically one or more of the set treatment parameters based on the predicted probability or the result. For example, the predicted probability or the result may be compared to pre-set thresholds, reference probabilities or results, classes of reference probabilities or results or the like which may be stored at a place accessible to the computer system, e.g. look-up table, a storage, or the like. Depending on this comparison, a pump rate may automatically be lowered, a treatment time prolonged, and so on. The extent to which this happens may be stored and, e.g., be linked to specific probabilities or results.

In several embodiments, the predicted probability or the result of the assessment may trigger outputting measures or advices as to how to deal with the fistula state assumed based on the probability or result, e.g. via a printer, a monitor, to a computer system, a hand-held apparatus like a smart phone, and the like.

The measures or advice may comprise a suggestion such as a surgical intervention, a check-up at the specialist, a stenting procedure of the fistula, and so on. A look-up table or a storage may be provided that allots particular measures to particular probabilities or results.

In some embodiments, the control unit is programmed to output suggestions regarding medical or surgical treatment based on the predicted probability or the result. This may be another step of the method according to the invention as well.

Optionally, the method steps described herein may be carried out in the above-mentioned order, with a time overlap or simultaneously.

In some embodiments, the hemodialysis treatment apparatus or any other device disclosed herein may determine the clearance or the reference clearance. The hemodialysis treatment apparatus or other device may include a control and evaluation unit (which may be the control unit disclosed herein), a device for influencing the dialysate composition at the dialyzer inlet to induce a change of the conductivity of the dialysate, a device for evaluating of the “response” that can be measured in the dialysate at the dialyzer outlet, and a device to gather a value for the clearance, which may be a value of urea or any other suitable substance, in particular electrolyte.

In particular embodiments, the hemodialysis treatment apparatus or any other device disclosed herein is configured for carrying out any method disclosed or mentioned herein for determining the clearance of urea or any other suitable substance, in particular electrolyte. It may, for example, comprise a device for measuring clearance via spectroscopy.

In certain embodiments, the hemodialysis treatment apparatus or any other device disclosed herein is configured for carrying out any method disclosed or mentioned herein for determining- or retrieving (from a memory, e.g.) the reference clearance as mentioned herein.

Some or all of the embodiments according to the present invention may comprise one, several or all of the advantages mentioned above and/or in the following.

As the present invention is capable of predicting the risk of failure of a vascular access, this may advantageously be extremely valuable for clinician and patients. Patient safety may be substantially increased.

As in some (or all) embodiments the computer-implemented method according to the present invention uses only data already collected by the hemodialysis treatment apparatus to develop an ML model for predicting the risk of failure of the fistula, it is relatively easy to implement the method with common hemodialysis treatment machines. This may help to save manpower and money.

Even with only a limited number of parameters considered and using a simple model such as logistic regression, it is possible to achieve reasonable performance. Therefore, it is possible to implement an ML model directly into the control unit or any other part of, or related to, the hemodialysis treatment apparatus, which allows for timely alert to be delivered and, therefore, to increase patient safety.

All the advantages achievable with the method according to the present invention can be in certain embodiments achieved undiminished by the apparatus and/or devices according to the present invention and vice versa.

In the following, the present invention is described based on preferred embodiments thereof with reference to accompanying drawing. However, the present invention is not to be limited to these embodiments. In the figures, same reference numerals denote identical or similar elements.

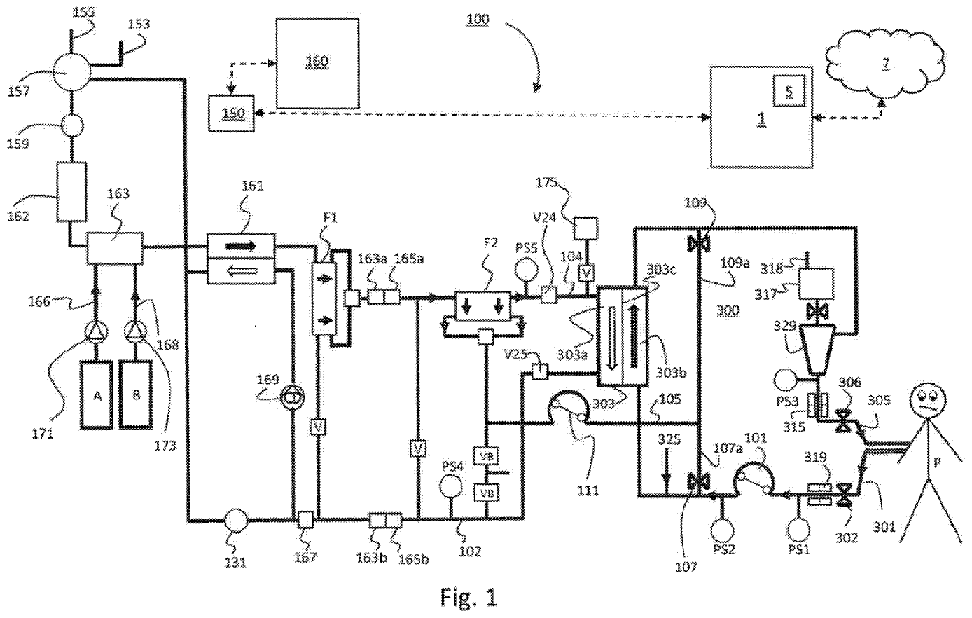

FIG. 1 shows, schematically simplified, a fluid line diagram of a hemodialysis treatment apparatus 100 according to the present invention in an exemplary embodiment.

The hemodialysis treatment apparatus 100, shown in an at least partially equipped state of use, is connected to an extracorporeal blood circuit 300, which may be connected for treatment to the vascular system of the patient P using double-needle access as shown in FIG. 1, or by using e.g. an additional Y-connector, for single-needle access or by using a central venous catheter or any other vascular access (or device, respectively), wherein said extracorporeal blood circuit 300 is optionally not part of the hemodialysis treatment apparatus 100, whereas it is in other embodiments. The blood circuit 300 may be present, optionally in sections thereof, in or on a blood cassette.

Pumps, actuators and/or valves in the area of the blood circuit 300 are in signal communication with the treatment apparatus 100 according to the present invention or with a control device 150, e.g. comprised by it, where required for controlling it or controlling it in a closed-loop manner.

The blood circuit 300 comprises (or is connected to) an arterial patient tubing clamp 302 and an arterial connection needle of an arterial section or of an arterial patient line, blood withdrawal line or first line 301. The blood circuit 300 also comprises (or is connected to) a venous patient tubing clamp 306 and a venous connection needle of a venous section, a venous patient line, blood return line or second line 305.

A blood pump 101 is provided in or at the first line 301, an optional substitute fluid pump 111 is connected to, if applicable, a dialysis liquid inlet line 104 for conveying fresh dialysis liquid, which may be filtered in an optional further filter stage F2 (substitute fluid). A substitute fluid line 105 may be fluidically connected to the dialysis liquid inlet line 104. Using the optional substitute fluid pump 111, substitute fluid may be introduced by pre-dilution, via a pre-dilution valve 107, or by post-dilution, via a post-dilution valve 109, via associated lines 107a or 109a into line sections, for example, into the arterial line section 301 or into the venous line section 305 (here between a blood chamber 303b of a blood filter 303 and a venous air separation chamber or venous bubble trap 329) of the blood circuit 300.

The blood filter 303 comprises the blood chamber 303b connected to the arterial line section 301 and to the venous line section 305. A dialysis liquid chamber 303a of the blood filter 303 is connected to the dialysis liquid inlet line 104 leading to the dialysis liquid chamber 303a and to a dialysate outlet line 102, leading away from the dialysis liquid chamber 303a, which dialysate outlet line 102 guides dialysate, i.e. spent dialysis liquid. Suitable connectors at the dialysis liquid inlet line 104 or at the dialysate outlet line 102 on the one hand and at the dialysate ports on the other, which may be, in particular releasably, connected to each other serve for this purpose.

Dialysis liquid chamber 303a and blood chamber 303b are separated from each other by a mostly semi-permeable membrane 303c. It represents the partition between the blood side with the extracorporeal blood circuit 300 and the machine side with the dialysis liquid or dialysate circuit, which is shown in FIG. 1 to the left of the membrane 303c.

The arrangement of FIG. 1 further comprises a valve V24 arranged in the dialysis liquid inlet line 104 upstream of the blood filter 303, but downstream of a pressure sensor PS5. It further comprises a valve V25 arranged in the dialysate outlet line 102, downstream of the blood filter 303, but upstream of another pressure sensor PS4.

The arrangement of FIG. 1 comprises an optional detector 315 for detecting air and/or blood. The arrangement in FIG. 1 further comprises one or two optional pressure sensors PS1 (upstream of the blood pump 101) and PS2 (downstream of the blood pump 101), it measures the pressure upstream of the blood filter 303 (“pre-hemofilter”) at the points shown in FIG. 1. Further pressure sensors may be provided, e.g. the pressure sensor PS3 downstream of venous bubble trap 329.

Blood exiting the blood filter 303 flows through the optional venous bubble trap 329, which may comprise a de-aeration device 318 and be in fluid communication with the pressure sensor PS3.

An optional single-needle chamber 317 is used in FIG. 1 as a buffer and/or compensating reservoir in a single-needle in which the patient is connected to the extracorporeal blood circuit 300 using only one of the two blood lines 301, 305.

The arrangement of FIG. 1 also comprises an optional detector 319 for detecting air bubbles and/or blood.

An addition site 325 for Heparin or for other anticoagulants may optionally be provided.

On the left in FIG. 1, there is shown an mixing device 163, which provides a predetermined mixture for the respective solution from the containers A (for A-concentrate via concentrate supply 166) and B (for B-concentrate via concentrate supply 168) for use by the hemodialysis treatment apparatus 100. The solution contains water from the water source 155 (on-line, e.g. as reverse osmosis water or from bags) which is heated, for example, in the heating device 162.

A pump 171, which may be referred to as concentrate pump or sodium pump, is in fluid communication with the mixing device 163 and a source of sodium, for instance the container A, and/or conveys out of it. An optional pump 173, associated with container B, for instance for bicarbonate, can also be seen.

Furthermore, FIG. 1 shows an outlet 153 for the effluent. An optional heat exchanger 157 and a first flow pump 159, which is suitable for degassing, complete the arrangement shown.

The pressure sensor PS4 downstream of the blood filter 303 on the water side, but preferably upstream of the ultrafiltration pump 131 in the dialysate outlet line 102 may be provided for measuring the filtrate pressure or membrane pressure of the blood filter 303.

The exemplary arrangement shown in FIG. 1 depicts the control device 150. The latter may be in a wired or wireless signal connection with any of the components mentioned herein-especially or in particular with the blood pump 101—to control or control in a closed-loop manner the hemodialysis treatment apparatus 100.

By using the device for on-line mixing of the dialysis liquid, a variation of its sodium content, controlled by the control device 150, is possible within certain limits. For this purpose, in particular the measurement values determined by the conductivity sensors 163a, 163b may be taken into account. Should an adjustment of the sodium content of the dialysis liquid (sodium concentration) or of the substitute fluid turn out to be necessary or desired, this can be done by adjusting the conveying rate of the sodium pump 171.

In addition, the treatment apparatus 100 comprises devices for conveying fresh dialysis liquid and dialysate. A first valve, such as valve V24, may be provided between the first flow pump 159 and the blood filter 303, which first valve opens or closes the inflow towards the blood filter 303 at the inlet side. A second, optional flow pump 169 which conveys dialysate towards the outlet 153 is provided, e.g., downstream of the blood filter 303. A second valve, such as valve V25, may be provided between the blood filter 303 and the second flow pump 169, which second valve opens or closes the outflow at the outlet side.

Furthermore, the treatment apparatus 100 optionally comprises a device 161 for balancing the flow flowing into and out of the dialyzer 303 on the machine side. The device 161 for balancing is preferably arranged in a line section between the first flow pump 159 and the second flow pump 169.

The treatment apparatus 100 further comprises ultrafiltration pump 131 for the precise removal of a volume of liquid from the balanced circuit, as preset by the user and/or by the control device 150.

Sensors such as the optional conductivity sensors 163a, 163b serve to determine the conductivity, which in some embodiments is temperature-compensated, as well as the liquid flow upstream and downstream of the dialyzer 303.

Temperature sensors 165a, 165b may be provided individually or in groups.

Temperature values supplied by them may be used to determine a temperature-compensated conductivity.

An optional compressed air source 175, for example in the form of a compressor, may be provided at the machine side upstream of the blood filter 303.

A leakage sensor 167 is optionally provided. It may alternatively be provided at another place.

Further flow pumps in addition to or alternatively to, e.g., the one with the reference numeral 169 may also be provided.

A number of optional valves are each denoted with V in FIG. 1; by-pass valves with VB.

In some embodiments, the control device 150 determines the electrolyte balance and/or liquid balance based on the measurement values from the aforementioned optional sensors.

Filters F1 and F2 can be connected in series.

Even when using non-pure water, the filter F1 exemplarily serves herein to generate sufficiently pure dialysis liquid by the mixing device 163, which then flows through the blood filter 303, e.g., using the countercurrent principle.

The filter F2 exemplarily serves herein to generate sterile or sufficiently filtered substitute fluid from the sufficiently pure dialysis liquid leaving the first filter F1, by filtering e.g. pyrogenic substances. Said substitute fluid may then be safely added to the extracorporeally flowing blood of the patient and thus ultimately to the patient's body.

The treatment apparatus 100 is optionally shown in FIG. 1 as an apparatus capable of performing hemo(dia)filtration. However, hemodialysis treatment apparatuses that do not perform hemodiafiltration are also encompassed by the present invention.

The arrows shown in FIG. 1 generally indicate the flow direction.

Furthermore, the control device 150 is, for the purpose of retrieving or transmitting data, for example adjustable settings and/or predicted data, in wired, or preferably wireless, signal communication (generally shown as dashed double arrows in FIG. 1) with the computer system 1 according to the present invention, which in the example of FIG. 1 may be physically separated from the hemodialysis treatment apparatus 100. The computer system 1 may comprise a calculation device 5 for executing the calculation steps, where applicable. Additionally, the computer system 1 may be connected in mutual signal communication with a central unit or cloud 7, from which e.g. input data may be retrieved. The central unit or cloud 7 may in some embodiments be or comprise the digital storage medium according to the present invention, or vice versa.

The control device 150 may be prepared to receive adjustable settings transmitted by signal transmission from the computer system 1. Alternatively or additionally, it may be programmed to control or control in a closed-loop manner the hemodialysis treatment apparatus 100 based on the adjustable settings transmitted via signal transmission.

The hemodialysis treatment apparatus 100 according to the present invention in the example of FIG. 1 further comprises a display 160, which is optionally provided and embodied for interacting with and/or informing the user. The display 160 may be designed as output device and/or as input device.

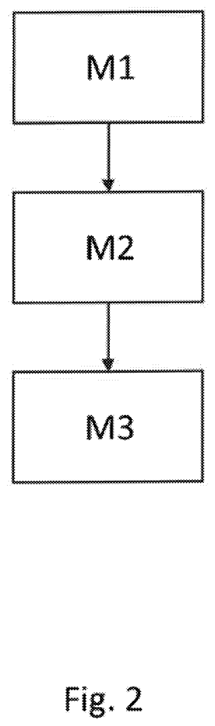

FIG. 2 shows in an exemplary embodiment a flowchart of a computer-implemented method according to the present invention for predicting a patient's (P) fistula failure or for assessing the probability of the fistula to fail, e.g., within a preset time period or in the course of pre-determined number of treatment sessions.

As a first method step M1 the computer-implemented method comprises the inputting of a plurality of parameter values into a computer system 1 or a component thereof. Some of these values have been measured during one or more dialysis treatment sessions while treating the patient P using a hemodialysis treatment apparatus, e.g. the one of FIG. 1, or in connection with such dialysis treatment sessions, respectively.

Alternatively or additionally, some of these values are or have been calculated based on, or derived from, values that have been measured during one or more dialysis treatment sessions while treating the patient P using a hemodialysis treatment apparatus 100.

The values of the parameters which are input comprise or consist of, at least two or more of:
clearance,
sum pressure,
clearance/blood flow-ratio, or
clearance/reference clearance-ratio.

The clearance is the capacity of the dialyzer 300 to clean the blood of urea or the urea removal rate and can be calculated based on urea measurements, preferably in as a in-vivo clearance measurement. For example, the Online Clearance Monitoring (OCM®) system by Fresenius Medical Care, Germany, integrated, e.g., into their therapy system 5008, intradialytically measures the effective in-vivo urea clearance achieved during the treatment session.

The sum pressure is the sum of the, e.g. simultaneously, measured arterial pressure and venous pressure, e.g. the pressure measured by sensors PS1 or PS2 (FIG. 1) plus the pressure measured, preferably simultaneously, by sensor PS3.

The clearance/blood flow-ratio is the clearance divided by the blood flow or vice versa, and the clearance/reference clearance-ratio is the clearance divided by a reference clearance, or vice versa.

As a second method step M2 the computer-implemented method encompasses predicting or assessing the estimated probability of an event predefined as a fistula failure within a predetermined time period. Thereby, the time period may start as of the time of the prediction or as of the time of executing/carrying out this method. Alternatively, an event may be predefined as a fistula failure within a predetermined number of, preferably subsequent, treatment sessions of the patient P.

In some embodiments of the computer-implemented method according to the present invention, the dynamic of the parameters during a treatment session of interest is not taken into account, but, for example, the median value of said parameter values measured during or calculated with respect to the treatment session.

For predicting the probability an artificial intelligence model, e.g., a machine learning model, may be used. Additionally or alternatively, a logistic regression model or a gradient boosting framework based on decision tree classifiers may be used.

The predicted probability may be considered the target variable or the dependent variable in statistics. A target variable is the outcome one aims to predict or explain using, e.g., a machine learning model. A target variable is the variable that is estimated or classified based on the input values.

Eventually, outputting the predicted probability or the result of the assessment is encompassed by the computer-implemented method as method step M3.

The probability or the result of the assessment may be output, for example to a printer, to a handheld device of any kind, e.g. a smartphone, a tablet etc. Additionally or alternatively, it may be output to the hemodialysis treatment apparatus 100.

Also, outputting suggestions regarding medical or surgical treatment based on the predicted probability or the result may be encompassed.

The present invention is not limited to the embodiments described herein. These are for illustrative purposes only.

It will be appreciated that the execution of the various machine-implemented processes and steps described herein may occur via the execution, by one or more respective processors, of processor-executable instructions stored on one or more tangible, non-transitory computer-readable mediums (such as random access memory (RAM), read-only memory (ROM), programmable read-only memory (PROM), and/or another electronic memory mechanism). Thus, for example, operations performed by various components as discussed herein may be carried out according to instructions stored on and/or applications installed on one or more respective computing devices.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present application covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### REFERENCE NUMERALS

1 computer system
5 calculation device
7 central unit; cloud
100 hemodialysis treatment apparatus
101 blood pump
102 dialysate outlet line
104 dialysis liquid inlet line
105 substitute fluid line
107 predilution valve
107a line associated with the predilution valve
109 postdilution valve
109a line associated with the postdilution valve
111 substitute fluid pump
131 ultrafiltration pump
150 control device
153 outlet for effluent
155 water source
157 heat exchanger
159 first flow pump
160 display
161 device for balancing
162 heating device
163 mixing device
163a conductivity sensor
163b conductivity sensor
165a temperature sensor
165b temperature sensor
166 concentrate supply
167 leakage sensor
168 concentrate supply
169 second flow pump
171 pump; sodium pump
173 pump; bicarbonate pump
175 compressed air source
300 extracorporeal blood circuit
301 first line; arterial line section; blood withdrawal line; arterial patient line
302 arterial patient tubing clamp
303 blood filter or dialyzer
303a dialysis liquid chamber
303b blood chamber
303c semi-permeable membrane
305 second line; venous line section; blood return line; venous patient line
306 venous patient tubing clamp
315 detector for detecting air bubbles and/or blood
317 single-needle chamber
318 de-aeration device
319 detector for detecting air bubbles and/or blood
325 addition site for anticoagulants, e.g. Heparin
329 venous air separation chamber; venous bubble trap (optional)
A container for A-concentrate; sodium
B container for B-concentrate; bicarbonate
F1 filter
F2 further filter level or stage
M1 to M3 method steps
P patient
PS1 arterial pressure sensor (optional)
PS2 arterial pressure sensor (optional)
PS3 pressure sensor (optional)
PS4 pressure sensor (optional)
PS5 pressure sensor
V valves
V24 valve
V25 valve
VB bypass valve

1. A hemodialysis system, comprising:
a hemodialysis treatment machine configured to provide a hemodialysis treatment for a patient, wherein the hemodialysis treatment includes using a dialyzer to remove urea from extracorporeal blood of the patient, and wherein the extracorporeal blood is obtained from the patient via a vascular access device of the patient;
one or more measuring devices configured to measure one or more measured values corresponding to the hemodialysis treatment; and
a computer system configured to:
obtain a plurality of parameter values, wherein the plurality of parameter values include the one or more measured values and/or one or more calculated values calculated from the one or more measured values;
predict a probability of failure of the vascular access device of the patient with a predetermined time period or within a predetermined number of treatment sessions; and
output the predicted probability.

2. The hemodialysis system according to claim 1, wherein the plurality of parameter values comprise two or more of the following parameters:
clearance, wherein the clearance corresponds to a capacity of the dialyzer to clean the extracorporeal blood of urea or a urea removal rate;
a sum pressure, wherein the sum pressure corresponds to a sum of arterial pressure and venous pressure;
a clearance/blood flow-ratio, wherein the clearance/blood flow-ratio corresponds to the clearance divided by blood flow or vice versa; or
a clearance/reference clearance-ratio, wherein the clearance/reference clearance ratio corresponds to the clearance divided by a reference clearance, or vice versa.

3. The hemodialysis system according to claim 1, wherein the plurality of parameter values include median values.

4. The hemodialysis system according to claim 1, wherein the predicted probability is output to a printer, a display, a handheld device, and/or to the hemodialysis treatment machine.

5. The hemodialysis system according to claim 1, wherein predicting the probability comprises using an artificial intelligence model.

6. The hemodialysis system according to claim 5, wherein predicting the probability comprises using a logistic regression model or a gradient boosting framework based on decision tree classifiers.

7. The hemodialysis system according to claim 1, wherein the computer system is further configured to:
output suggestions regarding medical or surgical treatment based on the predicted probability.

8. A vascular access monitoring method for a hemodialysis system, comprising:
providing, by a hemodialysis treatment machine, a hemodialysis treatment for a patient, wherein the hemodialysis treatment includes using a dialyzer to remove urea from extracorporeal blood of the patient, and wherein the extracorporeal blood is obtained from the patient via a vascular access device of the patient;
measuring, by one or more measuring devices, one or more measured values corresponding to the hemodialysis treatment;
obtaining, by a computer system, a plurality of parameter values, wherein the plurality of parameter values include the one or more measured values and/or one or more calculated values calculated from the one or more measured values;
predicting, by the computer system, a probability of failure of the vascular access device of the patient with a predetermined time period or within a predetermined number of treatment sessions; and
outputting, by the computer system, the predicted probability.

9. The method according to claim 8, wherein the plurality of parameter values comprise two or more of the following parameters:
clearance, wherein the clearance corresponds to a capacity of the dialyzer to clean the extracorporeal blood of urea or a urea removal rate;
a sum pressure, wherein the sum pressure corresponds to a sum of arterial pressure and venous pressure;
a clearance/blood flow-ratio, wherein the clearance/blood flow-ratio corresponds to the clearance divided by blood flow or vice versa; or
a clearance/reference clearance-ratio, wherein the clearance/reference clearance ratio corresponds to the clearance divided by a reference clearance, or vice versa.

10. The method according to claim 8, wherein the plurality of parameter values include median values.

11. The method according to claim 8, wherein the predicted probability is output to a printer, a display, a handheld device, and/or to the hemodialysis treatment machine.

12. The method according to claim 8, wherein predicting the probability comprises using an artificial intelligence model.

13. The method according to claim 12, wherein predicting the probability comprises using a logistic regression model or a gradient boosting framework based on decision tree classifiers.

14. The method according to claim 8, further comprising:
outputting, by the computer system, suggestions regarding medical or surgical treatment based on the predicted probability.

15. One or more non-transitory computer-readable mediums having processor-executable instructions stored thereon for vascular access monitoring for a hemodialysis system, wherein the processor-executable instructions when executed, facilitate performance of the following:
providing, by a hemodialysis treatment machine, a hemodialysis treatment for a patient, wherein the hemodialysis treatment includes using a dialyzer to remove urea from extracorporeal blood of the patient, and wherein the extracorporeal blood is obtained from the patient via a vascular access device of the patient;
measuring, by one or more measuring devices, one or more measured values corresponding to the hemodialysis treatment;
obtaining, by a computer system, a plurality of parameter values, wherein the plurality of parameter values include the one or more measured values and/or one or more calculated values calculated from the one or more measured values;
predicting, by the computer system, a probability of failure of the vascular access device of the patient with a predetermined time period or within a predetermined number of treatment sessions; and
outputting, by the computer system, the predicted probability.

16. The one or more non-transitory computer-readable mediums according to claim 15, wherein the plurality of parameter values comprise two or more of the following parameters:
clearance, wherein the clearance corresponds to a capacity of the dialyzer to clean the extracorporeal blood of urea or a urea removal rate;
a sum pressure, wherein the sum pressure corresponds to a sum of arterial pressure and venous pressure;
a clearance/blood flow-ratio, wherein the clearance/blood flow-ratio corresponds to the clearance divided by blood flow or vice versa; or
a clearance/reference clearance-ratio, wherein the clearance/reference clearance ratio corresponds to the clearance divided by a reference clearance, or vice versa.

17. The one or more non-transitory computer-readable mediums according to claim 15, wherein the plurality of parameter values include median values.

18. The one or more non-transitory computer-readable mediums according to claim 15, wherein the processor-executable instructions, when executed, further facilitate performance of the following:
outputting, by the computer system, suggestions regarding medical or surgical treatment based on the predicted probability.

19. The one or more non-transitory computer-readable mediums according to claim 15, wherein predicting the probability comprises using an artificial intelligence model.

20. The one or more non-transitory computer-readable mediums according to claim 15, wherein predicting the probability comprises using a logistic regression model or a gradient boosting framework based on decision tree classifiers.
